(19) United States
(12) Patent Application Publication
(10) Pub. No.: US 2011/0021402 A1
(43) Pub. Date: Jan. 27, 2011

(54) Detergent Composition

(75) Inventors: Ming Tang, Beijing (CN); Hiroshi Oh, Cincinnati, OH (US); Sen Liu, Beijing (CN); Victor Manuel Arredondo, West Chester, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(73) Assignee: The Procter & Gamble Company, Cincinnati, OH (US)

(21) Appl. No.: 12/895,963

(22) Filed: Oct. 1, 2010

Related U.S. Application Data
(63) Continuation of application No. PCT/US2010/041948, filed on Jul. 14, 2010.
(60) Provisional application No. 61/228,680, filed on Jul. 27, 2009.

(51) Int. Cl. C11D 7/32; C07C 309/04; C07C 317/04; C07F 9/09

(52) U.S. Cl. 510/218; 510/276; 510/347; 510/357; 562/109; 568/30; 558/208

(57) ABSTRACT

A solid detergent composition having the salt of an anionic surfactant and of a monopropanolamine counterion.

### CROSS REFERENCE TO RELATED APPLICATION(S)

This application is a Continuation of International Application No. PCT/US2010/041948, filed Jul. 14, 2010, which claims the benefit of U.S. Provisional Application Ser. No. 61/228,680, filed Jul. 27, 2009.

### FIELD OF THE INVENTION

The present invention relates to solid detergent composition comprising the salt of an anionic surfactant and of alkylamine counterion, in particular a monopropanolamine counterion.

### BACKGROUND OF THE INVENTION

Solid detergent compositions typically comprise anionic detergent surfactants. Surfactants perform many functions in a detergent composition such as the cleaning function and the sudsing function of the composition.

Many anionic surfactants commonly used in detergent are salts. A salt in the chemistry art is commonly defined as a product formed from the neutralization reaction of acids and bases. Salts are compounds composed of cations (positively charged ions) and anions (negative ions) so that the product is electrically neutral (without a net charge). There are several varieties of salts. Salts that produce hydroxide ions when dissolved in water are basic salts and salts that produce hydronium ions in water are acid salts. Neutral salts are those that are neither acid nor basic salts. Zwitterions contain an anionic centre and a cationic centre in the same molecule but are not considered to be salts. Examples include amino acids, many metabolites, peptides and proteins. When salts are dissolved in water, they are called electrolytes.

When the anionic surfactants are manufactured, they are often acidic. The acids are commonly neutralized with caustic (NaOH). The acid and caustic form a surfactant sodium salt. The sodium cations from the caustic are often referred to as “counterions” to the surfactant anions. In order to solubilize and stabilize these surfactants for shipping and future processing, water and other solvents are often added. The solution contains the anionic surfactant salt or electrolyte that includes the counterion. The solution containing surfactant and possibly a solvent that does not contain additional ingredients of a detergent may be referred to as a “paste”. Additional water and other solvents are typically also included in the final detergent composition.

The inventors have found that anionic surfactants neutralized with sodium counterion could still be improved in terms of sudsing benefit. In particular, there is a need to increase the quantity of suds that can be generated with a detergent composition. There is also a need to improve the suds mileage and the speed at which the suds is generated. Preferably, the sudsing benefit should be improved without substantially affecting the rinsing profile and/or the cleaning benefit of the detergent composition. There is also a need to fasten the dissolution rate of surfactant salts.

The inventors have surprisingly found that one or more of the above mentioned needs could be at least partially addressed by the use of detergent compositions, in particular detergent composition comprising a low level of builder(s) and/or surfactant(s), comprising the salt of an anionic surfactant and an alkylamine counterion, in particular the salt of an anionic surfactant and a monopropanolamine counterion.

### SUMMARY OF THE INVENTION

The present invention concerns a solid detergent composition comprising the salt of an anionic surfactant and of an alkylamine counterion, the detergent composition comprising less than 10% of phosphate and aluminosilicate builder(s) and/or the detergent composition comprising less than 15% of surfactant(s) and/or the salt of an anionic surfactant and of an alkylamine counterion comprising the salt of an anionic surfactant and of a monopropanolamine counterion.

According to an exemplary embodiment of the invention, the invention concerns a solid detergent composition comprising the salt of an anionic surfactant and of a monopropanolamine counterion.

According to an exemplary embodiment of the invention, the invention concerns a solid detergent composition comprising less than 10% of phosphate and aluminosilicate builder(s) and comprising the salt of an anionic surfactant and of an alkylamine counterion.

According to an exemplary embodiment of the invention, the invention concerns a solid detergent composition comprising less than 15% of surfactant(s) and comprising the salt of an anionic surfactant and of an alkylamine counterion.

Typically, the composition is in granulated form or in the form of tablet.

### DETAILED DESCRIPTION OF THE INVENTION

The solid detergent composition of the invention comprises at least one salt of an anionic surfactant and of an alkylamine counterion, in particular of a monopropanolamine counterion. The detergent composition may comprise at least 0.1%, or 0.2% or 0.3%, in particular from 0.4% to 90%, typically from 0.45% to 10%, or from 0.5% to 5% by weight of salts of an anionic surfactant and of an alkylamine counterion.

The detergent composition may comprise at least 0.1%, or 0.2%, or 0.3%, in particular from 0.4% to 90%, typically from 0.45% to 10%, or from 0.5% to 5% by weight of salts of an anionic surfactant and of a monopropanolamine counterion.

In the detergent composition of the invention, the ratio of salt(s) of an anionic surfactant and of an alkylamine counterion to the total amount of salt(s) of an anionic surfactant may be between 0.01 and 1, in particular may be between 0.02 and 0.9, and is typically between 0.03 and 0.3, or between 0.05 and 0.2, or even between 0.07 and 0.13.

In the detergent composition of the invention, the ratio of salt(s) of anionic surfactant and of monopropanolamine counterion to the total amount of salt(s) of anionic surfactant may be between 0.01 and 1, in particular may be between 0.02 and 0.9, and is typically between 0.03 and 0.3, or between 0.05 and 0.2, or even between 0.07 and 0.13.

The salt of an anionic surfactant and of an alkylamine counterion comprises any anionic surfactant and any alkylamine counterion, preferably any monopropanolamine counterion, which can form a salt together. Preferably, the alkylamine counterion and the anionic surfactant may be selected from the one disclosed below.

The Alkylamine Counterion

As used herein, the term alkylamine counterion refers to a counterion comprising at least one carbon atom and an amine. When they are neutralizing the anionic surfactant, the alkylamine forms the cationic, conjugate acid of the molecule. This cation may properly be referred to as a “alkylammonium” counterion, however, for the sake of consistency, the term “alkylamine” or “alkylamine counterion” will be used for purposes of this disclosure.

Preferably, the alkylamine counterion comprises at least 2 or even at least 3 carbon atoms. The alkylamine counterion may comprise 4 or 5 or more carbon atoms but will usually comprise 3 carbon atoms.

The alkylamine counterion preferably comprises a substitution of hydroxyl. Example of alkylamine counterion includes monoethanolamine (MEA), choline and propanolamine counterions.

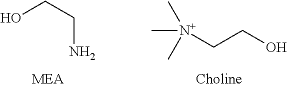

Preferably, the alkylamine counterion is a propanolamine counterion. As used herein, the term “propanolamine” refers to “monopropanolamines” only. When they are neutralizing the anionic surfactant, the propanolamine forms the cationic, conjugate acid of the molecule. This cation may properly be referred to as a “propanolammonium” counterion, however, for the sake of consistency, the term “propanolamine” or “propanolamine counterion” will be used for purposes of this disclosure. As used herein, “propanolamine counterion,” or the use of the term “propanolamine” in the context of an ion, refers to the cationic, conjugate acid of the propanolamine. Preferred propanolamines suitable for use as counterions in embodiments of the present disclosure include 2-amino-1-propanol (“2AP”), monoisopropanolamine (“MIPA” or “1-amino-2-propanol”), and 1-amino-3-propanol.

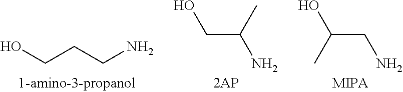

Typically, the propanolamines is selected from 2AP, MIPA, and combinations thereof. In particular the propanolamine is 2AP.

2AP can be made from glycerin, which is a feedstock made from naturally renewable resources. Other counterions, such as MEA, and MIPA are derived from petroleum, which is non-renewable. Further, because it is derived from oil, the price of MEA and MIPA are dependent on the price of oil, while 2AP is not.

2AP may exist in two enantiomeric forms. The conversion of glycerin to 2AP may provide a racemic mixture of the R and S isomers or may also provide it as a single enantiomer by selecting the appropriate chiral catalyst-ligand technology.

The Anionic Surfactant

The anionic surfactant which is part of the slat of an anionic surfactant and of an alkylamine counterion, may comprise any anionic surfactant which can be neutralized by a propanolamine counterion. In particular, the anionic surfactant may comprise anionic surfactants selected from alkyl ester sulfonate(s); linear, branched, and modified alkylbenzene sulfonate(s); C₁₀-C₁₈ alkyl alkoxy sulfates; C₁₀₋₂₀ primary, branched-chain and random alkyl sulfates; C₁₀-C₁₈ secondary (2,3) alkyl sulfates; C₁₀-C₁₈ alkyl alkoxy carboxylate(s); fatty acid(s); mid-chain branched alkyl sulfate(s); mid-chain branched alkyl alkoxy sulfate(s); alpha-olefin sulfonate(s); phosphate ester(s); and mixtures thereof.

### Alkyl Ester Sulfonate Surfactant (“MES”)

As used herein, “MES” refers to alkyl ester sulfonate surfactants, commonly used in methyl ester sulfonate form. MES surfactants useful herein include sulfonated fatty acid alkyl esters of the formula R—CH(SO3⁻)—COOR′, wherein R is, on the average, a C₆ to C₂₂ alkyl and R′ is on the average a C₁ to C₈ alkyl.

The hydrophobic portion of these sulfonated alkyl esters have the sulfonate group at the α-position, i.e., the sulfonate group is positioned at the carbon atom adjacent to the carbonyl group. The alkyl portion of the hydrophobic portion, which corresponds to the R portion of the sulfonated fatty acid alkyl esters, is on the average a C₆ to C₂₂ alkyl. Preferably, the alkyl portion of this hydrophobic portion, R, has a straight-chain of an average length C₈ to C₁₆ hydrocarbon particularly when R′ is methyl.

R′, forming the ester portion of the sulfonated alkyl esters, is on the average a C₁ to C₈ alkyl. Preferably, R′ is on the average a C₁ to C₆ alkyl, and most preferably a C₁ alkyl, i.e., methyl.

In one embodiment, the distribution is such that R is, on the average, a C₁₄ to C₁₆ alkyl (approximately, for example, a 95% C₁₄, 5% C₁₆ mixture) and R′ is methyl. In another embodiment, the distribution is such that R is, on the average, a C₁₂ to C₁₆ alkyl (approximately, for example, a 3% C₁₂, 28% C₁₄, 69% C₁₆ mixture) and R′ is methyl. In yet another embodiment, the distribution is such that R is, on the average, a C₁₀ to C₁₆ alkyl (approximately, for example, a 60% C₁₀, 35% C₁₂, 5% C₁₄ mixture) and R′ is methyl. In yet another embodiment, the distribution is such that R is, on the average, a C₁₂ to C₁₄ alkyl (approximately, for example, a 65% C₁₂, 30% C₁₄ mixture). In yet a further embodiment, blends of the aforementioned distributions of R and R′ may also be employed. In one embodiment, the methyl ester sulfonate has an average carbon length of about 16. In other embodiments, R′ could be ethyl (C₂), n-propyl & i-propyl (C₃), n-butyl, i-butyl (C₄), n-pentyl (C₅) and n-hexyl (C₆).

Methods of making alkyl ester surfactants neutralized with an alkali metal or an alkaline earth metal have been well described and are known to those skilled in art the art. See, for example, U.S. Pat. Nos. 4,671,900; 4,816,188; 5,329,030; 5,382,677; 5,384,422; 5,475,134; 5,587,500; 6,780,830. MES as such is commercially available from Huish.

### Linear, Branched, and Modified Alkylbenzene Sulfonate

Other suitable anionic surfactants useful herein include any of the conventional anionic surfactant types typically used in detergent products. These include alkyl benzene sulfonates as well as alkoxylated or non-alkoxylated alkyl sulfates.

Exemplary anionic surfactants are C₁₀₋₁₆ alkyl benzene sulfonates, preferably C₁₁₋₁₄ alkyl benzene sulfonates. In one embodiment, the alkyl group is linear and such linear alkyl benzene sulfonates are known as “LAS”. Alkyl benzene sulfonates, and particularly LAS, are well known in the art. Such surfactants and their preparation are described for example in U.S. Pat. Nos. 2,220,099 and 2,477,383. Preferred are the linear straight chain alkylbenzene sulfonates in which the average number of carbon atoms in the alkyl group is from about 11 to 14. Particularly, C₁₁-C₁₄, e.g., C₁₂, and LAS is a specific example of such surfactants.

Other exemplary alkylbenzene sulfonates include modified alkylbenzene sulfonate (MLAS) as discussed in WO 99/05243, WO 99/05242, WO 99/05244, WO 99/05082, WO 99/05084, WO 99/05241, WO 99/07656, WO 00/23549, and WO 00/23548.

MLAS may comprise a mixture, preferably consisting essentially of: (a) from about 15% to about 99%, preferably from about 15% to about 60%, more preferably from about 20% to about 40%, by weight of a mixture of branched alkylbenzene sulfonates having formula:

L(R1)(R2)-A-SO₃⁻

wherein L is an acyclic aliphatic moiety consisting of carbon and hydrogen, the L having two methyl termini and the L having no substituents other than A, R¹ and R²; and wherein the mixture of branched alkylbenzene sulfonates contains two or more, preferably at least three, optionally more, of the branched alkylbenzene sulfonates differing in molecular weight of the anion of the formula (I), and wherein the mixture of branched alkylbenzene sulfonates has a sum of carbon atoms in R¹, L and R² of from 9 to 15, preferably from 10 to 14; an average aliphatic carbon content, i.e., based on R′, L and R² and excluding A, of from about 10.0 to about 14.0, preferably from about 11.0 to about 13.0, more preferably from about 11.5 to about 12.5, carbon atoms; R¹ is C₁-C₃ alkyl, preferably C₁-C₂ alkyl, more preferably methyl; R² is selected from H and C₁-C₃ alkyl, preferably H and C₁-C₂ alkyl, more preferably H and methyl, more preferably H and methyl provided that in at least about 0.5, more preferably 0.7, more preferably 0.9 to 1.0 mole fraction of the branched alkylbenzene sulfonates, R² is H; A is a benzene moiety, typically A is the moiety-C₆H₄₋, with the SO₃⁻ moiety of Formula (I) in para-position to the L moiety, though in some proportion, usually no more than about 5%, preferably from 0 to 5% by weight, the SO₃⁻ moiety is ortho-to L; and (b) from about 1% to about 85%, preferably from about 40% to about 85%, more preferably from about 60% to about 80%, by weight of a mixture of nonbranched alkylbenzene sulfonates having formula:

Y-A-SO₃⁻

wherein A is as defined hereinbefore and Y is an unsubstituted linear aliphatic moiety consisting of carbon and hydrogen having two methyl termini, and wherein the Y has a sum of carbon atoms of from 9 to 15, preferably from 10 to 14, and the Y has an average aliphatic carbon content of from about 10.0 to about 14.0, preferably from about 11.0 to about 13.0, more preferably 11.5 to 12.5 carbon atoms; and wherein the modified alkylbenzene sulfonate surfactant mixture is further characterized by a ⅔-phenyl index of from about 160 to about 275, preferably from about 170 to about 265, more preferably from about 180 to about 255; and also preferably wherein the modified alkylbenzene sulfonate surfactant mixture has a 2-methyl-2-phenyl index of less than about 0.3, preferably less than about 0.2, more preferably less than about 0.1, more preferably still, from 0 to 0.05.

### C₁₀-C₁₈ Alkyl Alkoxy Sulfates

Another exemplary type of anionic surfactant includes ethoxylated alkyl sulfate surfactants. Such materials, also known as alkyl ether sulfates or alkyl polyethoxylate sulfates, are those which correspond to the formula:

R′—O—(C₂H₄O)ₙ—SO₃⁻

wherein R′ is a C₈-C₂₀ alkyl group and n is from about 1 to 20. In a specific embodiment, R′ is C₁₀-C₁₈ alkyl and n is from about 0.1 to 15. In another embodiment, n is from about 1 to 15. In more specific embodiments, R′ is a C₁₂-C₁₆, n is from about 1 to 6. In the disclosure herein, the designation “EOx” indicates that the alkoxy group is an ethoxy group, the integer “x” indicates the number of ethoxy groups in each chain.

The alkyl ether sulfates will generally be used in the form of mixtures comprising varying R′ chain lengths and varying degrees of ethoxylation. Frequently, though the average n value may be more than zero, such mixtures will inevitably also contain some non-ethoxylated alkyl sulfate materials, i.e., individual surfactant molecules of the above ethoxylated alkyl sulfate formula wherein n=0 for that particular molecule.

### C₁₀₋₂₀ Primary, Branched-Chain and Random Alkyl Sulfates

Alkyl sulfates may also be added separately to the compositions of this invention and used as or in any anionic surfactant component which may be present. Specific examples of alkyl sulfates surfactants are those produced by the sulfation of higher C₁₀-C₂₀ fatty alcohols. Conventional primary alkyl sulfate surfactants have the general formula:

ROSO₃⁻

wherein R is typically a linear C₁₀-C₂₀ alkyl group, which may be straight chain or branched chain. In specific embodiments, R is a C₁₀-C₁₅ alkyl, more specifically R is C₁₂-C₁₄.

### C₁₀-C₁₈ Secondary (2,3) Alkyl Sulfates

Another anionic surfactant useful herein includes secondary (2,3) alkyl sulfates having formulae CH₃—(CH₂)ₓ—CH(OSO₃⁻)—CH₃ or CH₃—(CH₂)_{y}—CH(OSO₃⁻)—CH₂—CH₃.

Non-limiting examples of a preferred secondary alkyl sulfate include the one where x is at least about 7, preferably at least about 9, and y is an integer of at least 8, preferably at least about 9.

### C₁₀-C₁₈ Alkyl Alkoxy Carboxylates

Another exemplary type of anionic surfactant includes ethoxylated alkyl carboxylate surfactants. Such materials, also known as alkyl ether carboxylates or alkyl polyethoxylate carboxylates, are those which correspond to the formula:

R′—O—(C₂H₄O)ₙ—COO⁻

wherein R′ is a C₈-C₂₀ alkyl group and n is an integer from about 1 to 20. In a specific embodiment, R′ is C₁₀-C₁₈ alkyl and/or n is from about 1 to 15. In more specific embodiments, R′ is a C₁₂-C₁₆ and/or n is from about 1 to 6.

The alkyl ether carboxylates will generally be used in the form of mixtures comprising varying R′ chain lengths and varying degrees of ethoxylation. Frequently such mixtures will inevitably also contain some non-ethoxylated alkyl carboxylate materials, i.e., surfactants of the above alkyl ether carboxylate formula wherein n=0.

### Fatty Acids

Fatty acids have the general formula:

RCOO⁻

wherein R is typically a C₉-C₂₁ alkyl group, which may be straight chain or branched chain. In specific embodiments, R is a C₉-C₁₇ alkyl, and more specifically R is C₁₁-C₁₅.

Exemplary fatty acids are selected from the group consisting of lauric acid, tridecylic acid, myristic acid, pentadecylic acid, palmitic acid, margaric acid, stearic acid, arachidic acid, phytanic acid, behenic acid, palmitoleic acid, oleic acid, elaidic acid, vaccenic acid, linoleic acid, cis-eleostearic acid, trans-eleosteric acid, linolenic acid, arachidonic acid and combinations thereof. Preferred fatty acids can be saturated or unsaturated. Unsaturated fatty acids typically having an iodine value from 15 to 25, preferably from 18 to 22 and a cis:trans isomer ratio from 1:1 to 200:1, preferably from 10:1 to 200:1.

Preferred sources of fatty acid are selected from the group consisting of coconut, soybean, tallow, palm, palm kernel, rapeseed, lard, sunflower, corn, safflower, canola, olive, peanut and combinations thereof.

### Mid-Chain Branched Alkyl Sulfates

Exemplary anionic surfactants include mid-chain branched alkyl sulfates as discussed in U.S. Pat. No. 6,020,303 and U.S. Pat. No. 6,060,443.

Mid-chain branched alkyl sulfates may comprise at least about 0.5%, preferably at least about 5%, more preferably at least about 10%, most preferably at least about 20%, by weight of longer alkyl chain, mid-chain branched surfactant compounds of the formula:

A^{b}-X-B

wherein:

(a) A^{b} is a hydrophobic C₉ to C₂₂ (total carbons in the moiety), preferably from about C₁₂ to about C₁₈, mid-chain branched alkyl moiety having: (1) a longest linear carbon chain attached to the -X-B moiety in the range of from 8 to 21 carbon atoms; (2) one or more C₁₋₃ alkyl moieties branching from this longest linear carbon chain; (3) at least one of the branching alkyl moieties is attached directly to a carbon of the longest linear carbon chain at a position within the range of position 2 carbon (counting from carbon #1 which is attached to the -X-B moiety) to position ω-2 carbon (the terminal carbon minus 2 carbons, i.e., the third carbon from the end of the longest linear carbon chain); and (4) the surfactant composition has an average total number of carbon atoms in the A^{b}-X moiety in the above formula within the range of greater than 14.5 to about 17.5 (preferably from about 15 to about 17);

(b) B is a hydrophilic moiety selected from sulfates; and

(c) X is selected from —CH₂— and —C(O)—.

Also preferred are mid-chain branched alkyl sulfates of the above formula wherein the A^{b} moiety does not have any quaternary substituted carbon atoms (i.e., 4 carbon atoms directly attached to one carbon atom).

Preferred mid-chain branched alkyl sulfates herein comprise longer alkyl chain, mid-chain branched surfactant compounds of the above formula wherein the A^{b} moiety is a branched primary alkyl moiety having the formula:

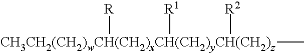

wherein the total number of carbon atoms in the branched primary alkyl moiety of this formula (Including the R, R¹, and R² branching) is from 13 to 19; R, R¹, and R² are each independently selected from hydrogen and C₁-C₃ alkyl (preferably methyl), provided R, R¹, and R² are not all hydrogen and, when z is 0, at least R or R¹ is not hydrogen; w is from 0 to 13; x is from 0 to 13; y is from 0 to 13; z is from 0 to 13; and w+x+y+z is from 7 to 13.

Also preferred mid-chain branched alkyl sulfates comprise longer alkyl chain, mid-chain branched surfactant compounds of the above formula wherein the A^{b} moiety is a branched primary alkyl moiety having the formula selected from:

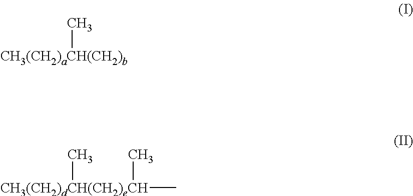

or mixtures thereof; wherein a, b, d, and e are integers, a+b is from 10 to 16, d+e is from 8 to 14 and wherein further when a+b=10, a is an integer from 2 to 9 and b is an integer from 1 to 8; when a+b=11, a is an integer from 2 to 10 and b is an integer from 1 to 9; when a+b=12, a is an integer from 2 to 11 and b is an integer from 1 to 10; when a+b=13, a is an integer from 2 to 12 and b is an integer from 1 to 11; when a+b=14, a is an integer from 2 to 13 and b is an integer from 1 to 12; when a+b=15, a is an integer from 2 to 14 and b is an integer from 1 to 13; when a+b=16, a is an integer from 2 to 15 and b is an integer from 1 to 14; when d+e=8, d is an integer from 2 to 7 and e is an integer from 1 to 6; when d+e=9, d is an integer from 2 to 8 and e is an integer from 1 to 7; when d+e=10, d is an integer from 2 to 9 and e is an integer from 1 to 8; when d+e=11, d is an integer from 2 to 10 and e is an integer from 1 to 9; when d+e=12, d is an integer from 2 to 11 and e is an integer from 1 to 10; when d+e=13, d is an integer from 2 to 12 and e is an integer from 1 to 11; when d+e=14, d is an integer from 2 to 13 and e is an integer from 1 to 12.

### Mid-Chain Branched Alkyl Alkoxy Sulfates

Still other exemplary anionic surfactants include mid-chain branched alkyl alkoxy sulfates as discussed in U.S. Pat. No. 6,008,181 and U.S. Pat. No. 6,020.303

Mid-chain branched alkyl alkoxy sulfates comprise from about 0.001% to about 100% of one or more (preferably a mixture of two or more) mid-chain branched primary alkyl alkoxylated sulfates having the formula:

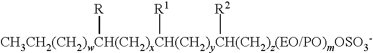

wherein the total number of carbon atoms in the branched primary alkyl moiety of this formula (Including the R, R¹, and R² branching, but not including the carbon atoms in the EO/PO alkoxy moiety) is from 14 to 20, and wherein further for this surfactant mixture the average total number of carbon atoms in the branched primary alkyl moieties having the above formula is within the range of greater than 14.5 to about 17.5 (preferably from about 15 to about 17); R, R¹, and R² are each independently selected from hydrogen and C₁-C₃ alkyl (preferably methyl), provided R, R¹, and R² are not all hydrogen and, when z is 1, at least R or R¹ is not hydrogen; w is an integer from 0 to 13; x is an integer from 0 to 13; y is an integer from 0 to 13; z is an integer of at least 1; w+x+y+z is from 8 to 14; and EO/PO are alkoxy moieties including for example ethoxy, propoxy, butoxy, etc, preferably selected from ethoxy, propoxy, and mixed ethoxy/propoxy groups, most preferably ethoxy, wherein m is at least about 0.01, preferably within the range of from about 0.1 to about 30, more preferably from about 0.5 to about 10, and most preferably from about 1 to about 5. It is to be recognized that the (EO/PO)ₘ moiety may be either a distribution with average degree of alkoxylation corresponding to m, or it may be a single specific chain with alkoxylation (e.g., ethoxylation and/or propoxylation) of exactly the number of units corresponding to m.

Preferably, the mid-chain branched alkyl alkoxy sulfates comprise a mixture of mid-chain branched primary alkyl alkoxylated sulfate surfactants, said mixture comprising at least about 5% by weight of two or more mid-chain branched primary alkyl alkoxylated sulfates having the formula:

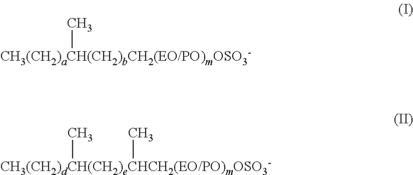

or mixtures thereof; wherein a, b, d, and e are integers, a+b is from 10 to 16, d+e is from 8 to 14 and wherein further when a+b=10, a is an integer from 2 to 9 and b is an integer from 1 to 8; when a+b=11, a is an integer from 2 to 10 and b is an integer from 1 to 9; when a+b=12, a is an integer from 2 to 11 and b is an integer from 1 to 10; when a+b=13, a is an integer from 2 to 12 and b is an integer from 1 to 11; when a+b=14, a is an integer from 2 to 13 and b is an integer from 1 to 12; when a+b=15, a is an integer from 2 to 14 and b is an integer from 1 to 13; when a+b=16, a is an integer from 2 to 15 and b is an integer from 1 to 14; when d+e=8, d is an integer from 2 to 7 and e is an integer from 1 to 6; when d+e=9, d is an integer from 2 to 8 and e is an integer from 1 to 7; when d+e=10, d is an integer from 2 to 9 and e is an integer from 1 to 8; when d+e=11, d is an integer from 2 to 8 and e is an integer from 1 to 9; when d+e=12, d is an integer from 2 to 11 and e is an integer from 1 to 10; when d+e=13, d is an integer from 2 to 12 and e is an integer from 1 to 11; when d+e=14, d is an integer from 2 to 13 and e is an integer from 1 to 12; wherein for this surfactant mixture the average total number of carbon atoms in the branched primary alkyl moieties having the above formulas is within the range of greater than 14.5 to about 17.5; and wherein EO/PO are alkoxy moieties, preferably selected from ethoxy, propoxy, and mixed ethoxy/propoxy groups, wherein m is at least about 0.01, preferably within the range of from about 0.1 to about 30, more preferably from about 0.5 to about 10, and most preferably from about 1 to about 5.

The mid-chain branched alkyl alkoxy sulfates may comprise compounds of formula:

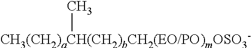

wherein: a is an integer from 2 to 11, b is an integer from 1 to 10, and a+b is 8 or 9; and EO/PO are alkoxy moieties, preferably selected from ethoxy, propoxy, and mixed ethoxy/propoxy groups, wherein m is at least about 0.01, preferably within the range of from about 0.1 to about 30, more preferably from about 0.5 to about 10, and most preferably from about 0.6 to about 5.

Also preferred herein are alkoxylated sulfate compounds of formula:

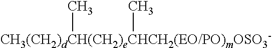

wherein: d and e are integers and d+e is 6 or 7; and wherein further when d+e=6, d is an integer from 2 to 5 and e is an integer from 1 to 4; when d+e=7, d is an integer from 2 to 6 and e is an integer from 1 to 5; and EO/PO are alkoxy moieties, preferably selected from ethoxy, propoxy, and mixed ethoxy/propoxy groups, wherein m is at least about 0.01, preferably within the range of from about 0.1 to about 30, more preferably from about 0.5 to about 10, and most preferably from about 0.6 to about 5.

### Alpha-Olefin Sulfonate

Other anionic surfactants useful in embodiments of the present disclosure include olefin sulfonates, which are compounds produced by the sulfonation of alpha-olefin by means of uncomplexed sulfur trioxide followed by neutralization of the acid reaction mixture under conditions such that sultones formed in the reaction are hydrolyzed to give corresponding hydroxyalkanesulfonates. The alpha-olefins from which the olefin sulfonates are derived are mono-olefins having from about 8 to about 24 carbon atoms, preferably from about 12 to about 16 carbon atoms. Preferably, they are straight chain olefins. Exemplary alpha-olefin sulfonates for use in the disclosure herein have the general formula:

R—CH═CH—CH2-SO₃⁻(2,3-alkenylsulfonate) or R—CH(OH)—CH2-CH2-SO₃⁻(3-hydroxy-alkanesulfonate,

where R is a linear or branched alkyl of about 8 to 20 carbon atoms. Examples of suitable alpha-olefins include 1-olefins such as 1-dodecene, 1-tetradecene, 1-hexadecene, 1-octadecene, 1-eicosene and 1-tetracosene.

### Phosphate Esters

Other anionic surfactants useful in embodiments of the present disclosure include phosphate esters. Phosphate esters are any materials of the general formula:

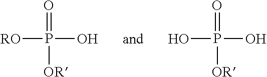

wherein R and R′ are C₆-C₂₀ alkyl or ethoxylated alkyl groups. Preferably R and R′ are of the general formula:

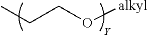

wherein the alkyl substituent is C₁₀-C₁₆ and Y is between 0 and about 4. Most preferably the alkyl substituent of that formula is C₁₀-C₁₆ and Y is between about 2 and about 4. Such compounds may be prepared by known methods from phosphorus pentoxide, phosphoric acid, or phosphorus oxy halide and alcohols or ethoxylated alcohols.

It will be appreciated that the formula depicted represent mono- and di-esters, and commercial phosphate esters will generally comprise mixtures of the mono- and di-esters, together with some proportion of tri-ester.

Salt of an anionic surfactant and of a monopropanolamine counterion may comprised salts selected from salts of formula R—CH(CH₃)-A-SO₃⁻:+NH₃CH₂(CH₃)CH₂OH with A being a benzene ring and R being a C₁₀-C₁₆ alkyl, R—CH₂(SO₃)COOCH₃⁻:+NH₃CH₂(CH₃)CH₂OH with R being a C₆-C₂₂ alkyl, ROSO₃⁻:⁺NH₃CH₂(CH₃)CH₂OH, with R being a C₁₀-C₂₀ alkyl, RO(CH₂CH₂O)ₓOSO₃⁻:⁺NH₃CH₂(CH₃)CH₂OH with R being a C₁₀-C₁₈ alkyl and x being between 0.1 and 15, and mixtures thereof.

Such salts includes in particular:

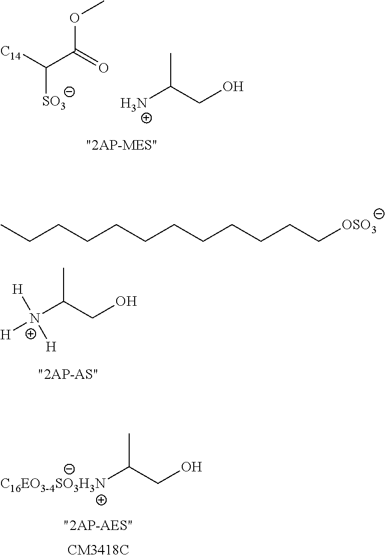

A surfactant paste comprising a salt of the invention may be prepared by adding a alkylamine counterion to an acid surfactant to neutralize the acid surfactant. Typically, particles comprising the salt of an anionic surfactant and of alkylamine counterion may be prepared by agglomeration or spray drying. Typically the particles comprising the salt of an anionic surfactant and of alkylamine counterion are dry-added to the solid detergent composition.

### Solid Detergent Composition

The solid detergent composition may be a laundry detergent composition or a dish washing detergent composition. Typically, the laundry detergent composition is formulated for use in an automatic washing machine or for hand-washing use.

The solid detergent composition may be in the form of an agglomerate, granule, flake, extrudate, bar, tablet or any combination thereof. The solid composition can be made by methods such as dry-mixing, agglomerating, compaction, spray drying, pan-granulation, spheronization or any combination thereof. The solid composition typically has a bulk density of from 300 g/l to 1,500 g/l, typically from 500 g/l to 1,000 g/l.

In addition to the salt(s) of anionic surfactant and of an alkylamine counterion, the solid detergent composition may comprise one or more adjunct ingredient(s). The precise nature of these additional adjunct components, and levels of incorporation thereof, will depend on the physical form of the composition and the nature of the operation for which it is to be used. Suitable adjunct materials include, but are not limited to, builder, additional surfactants, flocculating aid, chelating agents, dye transfer inhibitors, enzymes, enzyme stabilizers, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, processing aids, and/or pigments. In addition to the disclosure below, suitable examples of such other adjuncts and levels of use are found in U.S. Pat. Nos. 5,576,282, 6,306,812 B1 and 6,326,348 B1 that are incorporated by reference. Such one or more adjuncts may be present as detailed below:

SURFACTANTs—the detergent composition may comprise one or more additional surfactant(s). The additional surfactant(s) may be selected from nonionic surfactants, additional salts of anionic surfactants, cationic surfactants, ampholytic surfactants, zwitterionic surfactants, semi-polar nonionic surfactants and mixtures thereof.

The additional salt(s) of anionic surfactant(s) may be in the form of a salt of an anionic surfactant (as defined above, for example: alkyl ester sulfonate(s); linear, branched, and modified alkylbenzene sulfonate(s); C₁₀-C₁₈ alkyl alkoxy sulfates; C₁₀₋₂₀ primary, branched-chain and random alkyl sulfates; C₁₀-C₁₈ secondary (2,3) alkyl sulfates; C₁₀-C₁₈ alkyl alkoxy carboxylate(s); fatty acid(s); mid-chain branched alkyl sulfate(s); mid-chain branched alkyl alkoxy sulfate(s); alpha-olefin sulfonate(s); phosphate ester(s)) with a counterion other than an alkylamine counterion. The counterion may be a proton or a cation which provides charges neutrality such as a sodium, calcium, potassium, or magnesium, in particular a sodium cation.

The compositions of the invention may comprise non-ionic surfactant. Where present the non-ionic detersive surfactant(s) is generally present in amounts of from 0.5 to 20 wt %, or from 2 wt % to 4 wt %.

The non-ionic detersive surfactant can be selected from the group consisting of: alkyl polyglucoside and/or an alkyl alkoxylated alcohol; C₁₂-C₁₈ alkyl ethoxylates, such as, NEODOL® non-ionic surfactants from Shell; C₆-C₁₂ alkyl phenol alkoxylates wherein the alkoxylate units are ethyleneoxy units, propyleneoxy units or a mixture thereof; C₁₂-C₁₈ alcohol and C₆-C₁₂ alkyl phenol condensates with ethylene oxide/propylene oxide block polymers such as Pluronic® from BASF; C₁₄-C₂₂ mid-chain branched alcohols, BA, as described in more detail in U.S. Pat. No. 6,150,322; C₁₄-C₂₂ mid-chain branched alkyl alkoxylates, BAEx, wherein x=from 1 to 35, as described in more detail in U.S. Pat. No. 6,153,577, U.S. Pat. No. 6,020,303 and U.S. Pat. No. 6,093,856; alkylcelluloses as described in more detail in U.S. Pat. No. 4,565,647, specifically alkylpolyglycosides as described in more detail in U.S. Pat. No. 4,483,780 and U.S. Pat. No. 4,483,779; polyhydroxy fatty acid amides as described in more detail in U.S. Pat. No. 5,332,528, WO 92/06162, WO 93/19146, WO 93/19038, and WO 94/09099; ether capped poly(oxyalkylated) alcohol surfactants as described in more detail in U.S. Pat. No. 6,482,994 and WO 01/42408; and mixtures thereof.

The composition may comprise a cationic detersive surfactant. When present, typically the composition comprises from 0.1 wt % to 10 wt %, or from 1 wt % to 2 wt % cationic detersive surfactant.

Suitable cationic detersive surfactants are alkyl pyridinium compounds, alkyl quaternary ammonium compounds, alkyl quaternary phosphonium compounds, and alkyl ternary sulphonium compounds. The cationic detersive surfactant can be selected from the group consisting of: alkoxylate quaternary ammonium (AQA) surfactants as described in more detail in U.S. Pat. No. 6,136,769; dimethyl hydroxyethyl quaternary ammonium surfactants as described in more detail in U.S. Pat. No. 6,004,922; polyamine cationic surfactants as described in more detail in WO 98/35002, WO 98/35003, WO 98/35004, WO 98/35005, and WO 98/35006; cationic ester surfactants as described in more detail in U.S. Pat. No. 4,228,042, U.S. Pat. No. 4,239,660, U.S. Pat. No. 4,260,529 and U.S. Pat. No. 6,022,844; amino surfactants as described in more detail in U.S. Pat. No. 6,221,825 and WO 00/47708, specifically amido propyldimethyl amine; and mixtures thereof.

Highly preferred cationic detersive surfactants are mono-C₈₋₁₀ alkyl mono-hydroxyethyl di-methyl quaternary ammonium chloride, mono-C₁₀₋₁₂ alkyl mono-hydroxyethyl di-methyl quaternary ammonium chloride and mono-C₁₀ alkyl mono-hydroxyethyl di-methyl quaternary ammonium chloride. Cationic surfactants such as Praepagen HY (tradename Clariant) may be useful and may also be useful as a suds booster.

BUILDERS—The detergent composition may comprise one or more builders. When a builder is used, the subject composition will typically comprise from 1% to about 40%, typically from 2 to 25%, or even from about 5% to about 20%, or from 8 to 15% by weight of builder.

Builders include, but are not limited to, the alkali metal, ammonium and alkanolammonium salts of polyphosphates, alkali metal silicates, layered silicates, such as SKS-6 of Clariant®, alkaline earth and alkali metal carbonates, aluminosilicate builders, such as zeolite, and polycarboxylate compounds, ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, fatty acids, the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof.

The detergent compositions of the present invention may comprise from 0 to 20%, in particular less than 15% or 10%, for example less than 5% of zeolite. In particular, the detergent composition comprises from 0 to 20%, in particular less than 15% or 10%, for example less than 5% of aluminosilicate builder(s).

The detergent composition of the present invention may comprise from 0 to 20%, in particular less than 15% or 10%, for example less than 5% of phosphate builder(s). The solid detergent composition may comprise from 0 to 20%, in particular less than 15% or 10%, for example less than 5% of phosphate builder(s) and of aluminosilicate builder(s).

The total amount of phosphate builder(s), aluminosilicate builder(s), polycarboxylic acid builder(s), and additional silicate builder(s) in the detergent composition may be comprised from 0 to 25%, or even from 1 to 20%, in particular from 1 to 15%, especially from 2 to 10%, for example from 3 to 5%, by weight.

The detergent compositions of the present invention may comprise from 0 to 35%, in particular less than 25% or 20% or 15% or 10%, for example less than 5% of sodium carbonate.

The composition may further comprise any other supplemental builder(s), chelant(s), or, in general, any material which will remove calcium ions from solution by, for example, sequestration, complexation, precipitation or ion exchange. In particular the composition may comprise materials having at a temperature of 25° C. and at a 0.1M ionic strength a calcium binding capacity of at least 50 mg/g and a calcium binding constant log K Ca²⁺ of at least 3.50.

In the composition of the invention, the total amount of phosphate builder(s), aluminosilicate builder(s), polycarboxylic acid builder(s), additional silicate builder(s), and other material(s) having a calcium binding capacity superior to 50 mg/g and a calcium binding constant higher than 3.50 in the composition may be comprised from 0 to 25%, or even from 1 to 20%, in particular from 1 to 15%, especially from 2 to 10%, for example from 3 to 5%, by weight.

FLOCCULATING AID—The composition may further comprise a flocculating aid. Typically, the composition comprises at least 0.3% by weight of the composition of a flocculating aid. The composition may also be substantially free of flocculating aid. Typically, the flocculating aid is polymeric. Typically the flocculating aid is a polymer comprising monomer units selected from the group consisting of ethylene oxide, acrylamide, acrylic acid and mixtures thereof. Typically the flocculating aid is a polyethyleneoxide. Typically the flocculating aid has a molecular weight of at least 100,000 Da, in particular from 150,000 Da to 5,000,000 Da or even from 200,000 Da to 700,000 Da.

BLEACHING AGENT—The compositions of the present invention may comprise one or more bleaching agents. In general, when a bleaching agent is used, the compositions of the present invention may comprise from about 0.1% to about 50% or even from about 0.1% to about 25% bleaching agent by weight of the detergent composition. When present, suitable bleaching agents include bleaching catalysts, photobleaches for example Vitamin K3 and zinc or aluminium phtalocyanine sulfonate; bleach activators such as tetraacetyl ethylene diamine (TAED) and nonanoyloxybenzene sulphonate (NOBS); hydrogen peroxide; pre-formed peracids; sources of hydrogen peroxide such as inorganic perhydrate salts, including alkali metal salts such as sodium salts of perborate (usually mono- or tetra-hydrate), percarbonate, persulphate, perphosphate, persilicate salts and mixtures thereof, optionally coated, suitable coatings including inorganic salts such as alkali metal; and mixtures thereof.

The amounts of hydrogen peroxide source and peracid or bleach activator may be selected such that the molar ratio of available oxygen (from the peroxide source) to peracid is from 1:1 to 35:1, or even 2:1 to 10:1

FLUORESCENT WHITENING AGENT—The composition may contain components that may tint articles being cleaned, such as fluorescent whitening agent. When present, any fluorescent whitening agent suitable for use in a detergent composition may be used in the composition of the present invention. The most commonly used fluorescent whitening agents are those belonging to the classes of diaminostilbene-sulphonic acid derivatives, diarylpyrazoline derivatives and bisphenyl-distyryl derivatives.

Typical fluorescent whitening agents are Parawhite KX, supplied by Paramount Minerals and Chemicals, Mumbai, India; Tinopal® DMS and Tinopal® CBS available from Ciba-Geigy AG, Basel, Switzerland. Tinopal® DMS is the disodium salt of 4,4′-bis-(2-morpholino-4 anilino-s-triazin-6-ylamino)stilbene disulphonate. Tinopal® CBS is the disodium salt of 2,2′-bis-(phenyl-styryl)disulphonate.

FABRIC HUEING AGENTS—Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof.

POLYMERIC DISPERSING AGENTS—the compositions of the present invention can contain polymeric dispersing agents. These polymeric dispersing agents, if included, are typically at levels up to about 5%, typically from about 0.2% to about 2.5%, more typically from about 0.5% to about 1.5%. Suitable polymeric dispersing agents, include polymeric polycarboxylates, substituted (including quarternized and oxidized) polyamine polymers, and polyethylene glycols, such as: acrylic acid-based polymers having an average molecular of about 2,000 to about 10,000; acrylic/maleic-based copolymers having an average molecular weight of about 2,000 to about 100,000 and a ratio of acrylate to maleate segments of from about 30:1 to about 1:1; maleic/acrylic/vinyl alcohol terpolymers; polyethylene glycol (PEG) having a molecular weight of about 500 to about 100,000, typically from about 1,000 to about 50,000, more typically from about 1,500 to about 10,000; and water soluble or dispersible alkoxylated polyalkyleneamine materials.

POLYMERIC SOIL RELEASE AGENT—The compositions of the present invention can also contain polymeric soil release agent. polymeric soil release agent, or “SRA”, have hydrophilic segments to hydrophilize the surface of hydrophobic fibers such as polyester and nylon, and hydrophobic segments to deposit upon hydrophobic fibers and remain adhered thereto through completion of washing and rinsing cycles, thereby serving as an anchor for the hydrophilic segments. This can enable stains occurring subsequent to treatment with the SRA to be more easily cleaned in later washing procedures. Preferred SRA's include oligomeric terephthalate esters; sulfonated product of a substantially linear ester oligomer comprised of an oligomeric ester backbone of terephthaloyl and oxyalkyleneoxy repeat units and allyl-derived sulfonated terminal moieties covalently attached to the backbone; nonionic end-capped 1,2-propylene/polyoxyethylene terephthalate polyesters; an oligomer having empirical formula (CAP)₂ (EG/PG)₅ (T)₅ (SIP)ᵢ which comprises terephthaloyl (T), sulfoisophthaloyl (SIP), oxyethyleneoxy and oxy-1,2-propylene (EG/PG) units and which is typically terminated with end-caps (CAP), typically modified isethionates, as in an oligomer comprising one sulfoisophthaloyl unit, 5 terephthaloyl units, oxyethyleneoxy and oxy-1,2-propyleneoxy units in a defined ratio, typically about 0.5:1 to about 10:1, and two-end-cap units derived from sodium 2-(2-hydroxyethoxy)-ethanesulfonate; oligomeric esters comprising: (1) a backbone comprising (a) at least one unit selected from the group consisting of dihydroxy sulfonates, polyhydroxy sulfonates, a unit which is at least trifunctional whereby ester linkages are formed resulting in a branched oligomer backbone, and combinations thereof; (b) at least one unit which is a terephthaloyl moiety; and (c) at least one unsulfonated unit which is a 1,2-oxyalkyleneoxy moiety; and (2) one or more capping units selected from nonionic capping units, anionic capping units such as alkoxylated, typically ethoxylated, isethionates, alkoxylated propanesulfonates, alkoxylated propanedisulfonates, alkoxylated phenolsulfonates, sulfoaroyl derivatives and mixtures thereof. Preferred are esters of the empirical formula:

((CAP)ₐ(EG/PG)_{b}(DEG)_{c}PEG)_{d}(T)ₑ(SIP)_{f}(SEG)_{g}(B)ₕ)

wherein CAP, EG/PG, PEG, T and SIP are as defined hereinabove, DEG represents di(oxyethylene)oxy units, SEG represents units derived from the sulfoethyl ether of glycerin and related moiety units, B represents branching units which are at least trifunctional whereby ester linkages are formed resulting in a branched oligomer backbone, a is from about 1 to about 12, b is from about 0.5 to about 25, c is from 0 to about 12, d is from 0 to about 10, b+c+d totals from about 0.5 to about 25, e is from about 1.5 to about 25, f is from 0 to about 12; e+f totals from about 1.5 to about 25, g is from about 0.05 to about 12; h is from about 0.01 to about 10, and a, b, c, d, e, f, g, and h represent the average number of moles of the corresponding units per mole of the ester; and the ester has a molecular weight ranging from about 500 to about 5,000; and; cellulosic derivatives such as the hydroxyether cellulosic polymers available as METHOCEL® from Dow; the C₁-C₄ alkyl celluloses and C₄ hydroxyalkyl celluloses, see U.S. Pat. No. 4,000,093, issued Dec. 28, 1976 to Nicol et al., and the methyl cellulose ethers having an average degree of substitution (methyl) per anhydroglucose unit from about 1.6 to about 2.3 and a solution viscosity of from about 80 to about 120 centipoise measured at 20° C. as a 2% aqueous solution. Such materials are available as METOLOSE SM100® and METOLOSE SM200®, which are the trade names of methyl cellulose ethers manufactured by Shinetsu Kagaku Kogyo KK.

ENZYME—The composition of the invention may further comprise an enzyme. When present in the detergent composition, the enzymes may be present at levels from about 0.00001% to about 2%, from about 0.0001% to about 1% or even from about 0.001% to about 0.5% or 0.02% enzyme protein by weight of the composition.

Examples of suitable enzymes include, but are not limited to, hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof.

ENZYME STABILIZERS—Enzymes for use in detergents can be stabilized by various techniques. The enzymes employed herein can be stabilized by the presence of water-soluble sources of calcium and/or magnesium ions in the finished compositions that provide such ions to the enzymes. In case of aqueous compositions comprising protease, a reversible protease inhibitor, such as a boron compound, can be added to further improve stability.

CATALYTIC METAL COMPLEXES—The compositions of the invention may comprise catalytic metal complexes. When present, one type of metal-containing bleach catalyst is a catalyst system comprising a transition metal cation of defined bleach catalytic activity, such as copper, iron, titanium, ruthenium, tungsten, molybdenum, or manganese cations, an auxiliary metal cation having little or no bleach catalytic activity, such as zinc or aluminum cations, and a sequestrate having defined stability constants for the catalytic and auxiliary metal cations, particularly ethylenediaminetetraacetic acid, ethylenediaminetetra(methylenephosphonic acid) and water-soluble salts thereof. Such catalysts are disclosed in U.S. Pat. No. 4,430,243.

If desired, the compositions herein can be catalyzed by means of a manganese compound. Such compounds and levels of use are well known in the art and include, for example, the manganese-based catalysts disclosed in U.S. Pat. No. 5,576,282.

Cobalt bleach catalysts useful herein are known, and are described, for example, in U.S. Pat. No. 5,597,936; U.S. Pat. No. 5,595,967. Such cobalt catalysts are readily prepared by known procedures, such as taught for example in U.S. Pat. No. 5,597,936, and U.S. Pat. No. 5,595,967.

Compositions herein may also suitably include a transition metal complex of ligands such as bispidones (WO 05/042532 A1) and/or macropolycyclic rigid ligands—abbreviated as “MRLs”. As a practical matter, and not by way of limitation, the compositions and processes herein can be adjusted to provide on the order of at least one part per hundred million of the active MRL species in the aqueous washing medium, and will typically provide from about 0.005 ppm to about 25 ppm, from about 0.05 ppm to about 10 ppm, or even from about 0.1 ppm to about 5 ppm, of the MRL in the wash liquor.

Suitable transition-metals in the instant transition-metal bleach catalyst include, for example, manganese, iron and chromium. Suitable MRLs include 5,12-diethyl-1,5,8,12-tetraazabicyclo[6.6.2]hexadecane.

Suitable transition metal MRLs are readily prepared by known procedures, such as taught for example in WO 00/32601, and U.S. Pat. No. 6,225,464.

SOFTENING SYSTEM—the compositions of the invention may comprise a softening agent such as clay for softening through the wash. The composition may additionally comprise a charged polymeric fabric-softening boosting component.

COLORANT—the compositions of the invention may comprise a colorant, typically a dye or a pigment. Particularly, preferred dyes are those which are destroyed by oxidation during a laundry wash cycle. To ensure that the dye does not decompose during storage it is preferable for the dye to be stable at temperatures up to 40° C. The stability of the dye in the composition can be increased by ensuring that the water content of the composition is as low as possible. If possible, the dyes or pigments should not bind to or react with textile fibres. If the colorant does react with textile fibres, the colour imparted to the textiles should be destroyed by reaction with the oxidants present in laundry wash liquor. This is to avoid coloration of the textiles, especially over several washes. Particularly, preferred dyes include but are not limited to Basacid® Green 970 from BASF and Monastral blue from Albion.

The following examples are given by way of illustration only and therefore should not be construed to limit the scope of the invention.

### Examples

| | Example 1 | Example 2 | Example 3 | Example 4 |
|---|---|---|---|---|
| Component | Concentration (Weight percents) | | | |
| Sodium LAS | 12 | 14 | 8 | 5 |
| 2AP LAS* | 2 | 1 | | |
| Sodium AE3S | 2 | | | |
| Sodium AE1S | | 2 | | 3 |
| 2AP AE1S* | | | 4 | 2 |
| 2AP MES* | | 1 | | |
| Cationic surfactant | 0.7 | 0.8 | 0.6 | 0.5 |
| Non ionic surfactant | 0.3 | 0.3 | | |
| Zeolite | 4 | 3 | | |
| Sodium carbonate | 32 | 25 | 35 | 34 |
| Silicate | 3 | 4 | 7 | 8 |
| polymers | 12 | 11 | 15 | 14 |
| enzyme | 2 | 1.5 | 2.5 | 2.6 |
| Bleach system | 6 | 5 | 8 | 10 |
| Sodium sulfate | 15 | 24 | 12 | 15 |
| Water and miscelaneous | bal | bal | bal | bal |
| Ratio (Salt(s) of an | 0.125 | 0.111 | 0.333 | 0.200 |
| anionic surfactant and | | | | |
| of a monopropanile | | | | |
| conterion)/(Total | | | | |
| amount of salt(s) of | | | | |
| an anionic surfactant) | | | | |

*dry added particles.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A solid detergent composition comprising the salt of an anionic surfactant and of a monopropanolamine counterion.

2. The composition according to claim 1 comprising less than about 10% of phosphate builder and of aluminosilicate builder.

3. The composition according to claim 1 comprising less than about 15% of surfactant.

4. The composition according to claim 1 comprising the salt of an anionic surfactant and of a monopropanolamine counterion selected from 2-amino-1-propanol, 1-amino-2-propanol, 1-amino-3-propanol and mixtures thereof.

5. The composition according to claim 1 comprising the salt of a monopropanolamine counterion and of an anionic surfactant selected from alkyl ester sulfonate; linear, branched, and modified alkylbenzene sulfonate; C₁₀-C₁₈ alkyl alkoxy sulfates; C₁₀₋₂₀ primary, branched-chain and random alkyl sulfates; C₁₀-C₁₈ secondary (2,3) alkyl sulfates; C₁₀-C₁₈ alkyl alkoxy carboxylate; fatty acid; mid-chain branched alkyl sulfate; mid-chain branched alkyl alkoxy sulfate; alpha-olefin sulfonate; phosphate ester; and mixtures thereof.

6. The composition according to claim 1 comprising at least about 0.4% by weight of salt(s) of an anionic surfactant and of an alkylamine counterion.

7. The composition according to claim 1 wherein the ratio of salt of an anionic surfactant and of an alkylamine counterion to the total amount of salt of anionic surfactant is between about 0.03 and about 0.3.

8. A solid detergent composition comprising the salt of an anionic surfactant and of an alkylamine counterion, the detergent composition comprising less than about 10% of phosphate builder and of aluminosilicate builder.

9. The composition according to claim 8, comprising the salt of an anionic surfactant and of a monopropanolamine counterion.

10. The composition according to claim 8 comprising the salt of an anionic surfactant and of a monopropanolamine counterion selected from 2-amino-1-propanol, 1-amino-2-propanol, 1-amino-3-propanol and mixtures thereof.

11. A solid detergent composition comprising the salt of an anionic surfactant and of an alkylamine counterion, the detergent composition comprising less than 14% of surfactant.

12. The composition according to claim 11, comprising the salt of an anionic surfactant and of a monopropanolamine counterion.

13. The composition according to claim 11, comprising the salt of an anionic surfactant and of a monopropanolamine counterion selected from 2-amino-1-propanol, 1-amino-2-propanol, 1-amino-3-propanol and mixtures thereof.
